SEQUENCE LISTING (12 sequences)

SEQ ID NO: 1 - protein, 53 aa, Artificial Sequence
  TRS1
LYNRVAVQGDVVRELKAKKAPKEDVDAAVKQLLSLKAEYKEKTGQEYKPGNPP

SEQ ID NO: 2 - protein, 53 aa, Artificial Sequence
  TRS2
LYDEVAAQGEVVRKLKAEKSPKAKINEAVECLLSLKAQYKEKTGKEYIPGQPP

SEQ ID NO: 3 - protein, 49 aa, Artificial Sequence
  TRS3
LFDKVASQGEVVRKLKTEKAPKDQVDIAVQELLQLKAQYKSLIGVEYKP

SEQ ID NO: 4 - protein, 20 aa, Artificial Sequence
  Linker 1
AEIGQNISSNSSASILESKS

SEQ ID NO: 5 - protein, 25 aa, Artificial Sequence
  Linker 2
LSQSSDSSPTRNSEPAGLETPEAKV

SEQ ID NO: 6 - protein, 200 aa, Artificial Sequence
  EPRS
LYNRVAVQGDVVRELKAKKAPKEDVDAAVKQLLSLKAEYKEKTGQEYKPGNPPAEIGQNISSNSSASILESKSLYDEVAAQGEVVRKLKAEKSPKAKINEAVECLLSLKAQYKEKTGKEYIPGQPPLSQSSDSSPTRNSEPAGLETPEAKVLFDKVASQGEVVRKLKTEKAPKDQVDIAVQELLQLKAQYKSLIGVEYKP

SEQ ID NO: 7 - protein, 238 aa, Artificial Sequence
  COVID-19 RBD
FTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNF

SEQ ID NO: 8 - protein, 654 aa, Artificial Sequence
  COVID-19 S1
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIG

SEQ ID NO: 9 - protein, 103 aa, Artificial Sequence
  Heat-labile toxin B subunit(LTB)
APQTITELCSEYRNTQIYTINDKILSYTESMAGKREMVIITFKSGETFQVEVPGSQHIDSQKKAIERMKDTLRITYLTETKIDKLCVWNNKTPNSIAAISMKN

SEQ ID NO: 10 - protein, 158 aa, Artificial Sequence
  Bacterioferritin
MKGDTKVINYLNKLLGNELVAINQYFLHARMFKNWGLKRLNDVEYHESIDEMKHADRYIERILFLEGLPNLQDLGKLNIGEDVEEMLRSDLALELDGAKNLREAIGYADSVHDYVSRDMMIEILRDEEGHIDWLETELDLIQKMGLQNYLQAQIREEG

SEQ ID NO: 11 - protein, 471 aa, Artificial Sequence
  RSV F
QNITEEFYQSTCSAVSKGYLSALRTGWYTSVITIELSNIKENKCNGTDAKVKLIKQELDKYKNAVTELQLLMQSTPPTNNRARRGGSGGSGGSGFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELL

SEQ ID NO: 12 - protein, 104 aa, Artificial Sequence
  JEV Envelope domain III(ED3)
KGTTYGMCTEKFSFAKNPADTGHGTVVIELSYSGSDGPCKIPIVSVASLNDMTPVGRLVTVNPFVATSSANSKVLVEMEPPFGDSYIVVGRGDKQINHHWHKAG

(19) United States
(12) Patent Application Publication
(10) Pub. No.: US 2023/0041904 A1
(43) Pub. Date: Feb. 9, 2023

(54) METHOD FOR ENHANCING WATER SOLUBILITY OF TARGET PROTEIN BY WHEP DOMAIN FUSION

(71) Applicant: Industry-Academic Cooperation Foundation, Yonsei University, Seoul (KR)

(72) Inventors: Baik Lin SEONG, Seoul (KR); Jin Hee LEE, Seoul (KR); Young Seok KIM, Seoul (KR); Yu Cheol CHEONG, Seoul (KR)

(73) Assignee: Industry-Academic Cooperation Foundation, Yonsei University, Seoul (KR)

(21) Appl. No.: 17/787,277

(22) PCT Filed: Dec. 21, 2020
(86) PCT No.: PCT/KR2020/018837

§ 371 Date: Jun. 17, 2022

(30) Foreign Application Priority Data
Dec. 19, 2019 (KR) 10-2019-0171057

(51) Int. Cl. C12N 9/00; C12N 15/70; C07K 14/565; C07K 14/005

(52) U.S. Cl. CPC C12N 9/93; C12Y 601/01015; C12N 15/70; C07K 14/565; C07K 14/005; C07K 2319/40; C12N 2770/18022; C12N 2760/18522; C12N 2770/24122

(57) ABSTRACT

The present invention relates to a fusion protein for enhancing expression efficiency of a target protein. More specifically, the present invention relates to a glutamyl-prolyl-tRNA synthetase from human (hEPRS) WHEP domain (including WHEP domains TRS-1, TRS-2, and TRS-3 which locate at middle sites of the EPRS protein, and linkers connecting the three domains therethrough). When used as a fusion protein for expressing a target protein in E. coli, the hEPRS WHEP domain according to the present invention enhanced water solubility of the target protein.

### TECHNICAL FIELD

The present invention relates to a fusion protein for increasing the expression efficiency of a target protein. More particularly, the present invention relates to WHEP domains (including WHEP domains TRS-1, TRS-2 and TRS-3 located in the central area of an EPRS protein and linkers connecting the three domains) of human glutamyl-prolyl-tRNA synthetase (hEPRS). When the WHEP domains of hEPRS according to the present invention are used as fusion proteins for expression of a target protein in E. coli, the solubility of the target protein is improved.

### BACKGROUND ART

Generally, proteins produced by biotechnology may broadly be classified into pharmaceutical and research proteins such as immunomodulators, enzyme inhibitors, or hormones and industrial proteins such as diagnostic proteins or enzymes added for a specific reaction, and production process technology development and industrialization are being promoted mainly using these two types of proteins. Particularly, when a useful recombinant protein is produced using recombinant microorganism technology, because of its advantages such as well-known genetic information, construction of various vector systems, and rapid culture in a relatively cheap medium at a high concentration, E. coli is used for various purposes in research or industry.

In the production of recombinant proteins in E. coli, various expression vectors with a strong inducible promoter have been developed and used for the production of foreign proteins. However, in the case of using E. coli as a host cell, a protein to be manufactured may be frequently degraded by a proteolytic enzyme in E. coli to decrease yield, and particularly, it is known that this tendency is more pronounced in the expression of a polypeptide with a small molecular weight of 10 kDa or less. In addition, generally, since transcription and translation of a protein occur in E. coli at almost the same time, an insoluble aggregate (inclusion body) is frequently found in the overexpression of a recombinant protein, and in the case of polypeptides expressed as an aggregate, a folding intermediate may be non-selectively bound to other protein impurities (a chaperone, a ribosome, an initiation factor, etc.) in host cells by an intermolecular disulfide bond or hydrophobic interaction, thereby having a disadvantage of decreasing purity in the aggregate of a target polypeptide. In addition, to make the protein expressed as described above an active form, the protein has to undergo a refolding process in which the protein is dissolved using a denaturant such as guanidine hydrochloride or urea, and then diluted, but in this case, there is the problem of a decreased production yield in that the protein is not folded into an active form (Marston F A et al., Biochem J 240(1):1-12, 1986).

The inventors conducted research to confirm the solubility-enhancing ability of a WHEP domain as a fusion protein. Preferentially, TRS-1 and TRS-2 having only one WHEP domain, and multiple WHEP domains (referred to as EPRS) including three WHEP domains (TRS-1, TRS-2 and TRS-3) and linkers were manufactured. In addition, the ability of the three types of fusion proteins to enhance target protein solubility was confirmed. It was confirmed that the solubility-enhancing ability of the multiple WHEP domain among the three fusion proteins is most superior, and the present invention was completed.

To express conventionally known proteins that are soluble and difficult to express in E. coli, solubility was compared using TRS-1, TRS-2 or EPRS as a fusion protein and a green fluorescent protein (GFP) as a target protein. As a result, it was confirmed that, among the fusion proteins including the WHEP domains of hEPRS, EPRS most highly contributes to an increase in solubility. The solubility-enhancing effect caused by EPRS was additionally confirmed with other target proteins, such as interferon-beta (IFN-β), TEV protease, and heat-labile enterotoxin subunit B (LTB).

### DISCLOSURE

### Technical Problem

The present invention is directed to providing a peptide capable of enhancing the solubility and folding of a target protein using a WHEP domain as a fusion partner, and an expression vector or gene construct for producing a recombinant protein.

The present invention is also directed to providing a configuration of a WHEP domain capable of significantly increasing the solubility of a target protein.

The present invention is also directed to providing a recombinant microorganism which is transformed with the expression vector or into which a gene construct is inserted, and a method of producing a recombinant protein using the same.

### Technical Solution

The present invention provides a peptide for enhancing the expression efficiency of a target protein, which includes the sequence of a domain isolated from human glutamyl-prolyl tRNA synthetase (hEPRS).

According to one exemplary embodiment of the present invention, the domain may include TRS-1 (SEQ ID NO: 1) and TRS-2 (SEQ ID NO: 2), and most preferably, the domain includes TRS-1 (SEQ ID NO: 1) and TRS-2 (SEQ ID NO: 2).

The peptide of the present invention may further include a linker sequence to connect the sequences of the domains with each other, and the linker sequence may be represented by SEQ ID NO: 3.

In addition, the peptide may further include a tag sequence for enhancing the expression efficiency of a target protein.

In the present invention, the target protein may be one or more selected from the group consisting of an antigen, an antibody, a cell receptor, an enzyme, a structural protein, serum and a cell protein, and the antigen may be one or more selected from the group consisting of novel coronavirus (SARS-CoV-2), the hemagglutinin (HA) of influenza virus, respiratory syncytial virus (RSV) and Nipah virus. According to the most exemplary embodiment of the present invention, the antigen is RSV.

The present invention also provides a polynucleotide encoding the peptide.

According to one embodiment of the present invention, an expression vector which includes a polynucleotide encoding a target protein; and a polynucleotide encoding a peptide according to any one of claims 1 to 8 binding to the 5′-end of the polynucleotide encoding the target protein is provided.

According to another embodiment of the present invention, host cells transformed with the expression vector are provided, and the host cells may be E. coli.

The present invention provides a method of producing a soluble target protein, which includes: (A) preparing an expression vector including a polynucleotide encoding a target protein and a nucleotide encoding the peptide of any one of claims 1 to 8 binding to the 5′ end of the polynucleotide; (B) preparing a transformant by introducing the expression vector into host cells; and (C) culturing the transformant to induce the expression of a recombinant target protein and obtaining the target protein.

### ADVANTAGEOUS EFFECTS

A method of producing a recombinant protein using a WHEP domain according to the present invention as a fusion partner improves the solubility and expression rate of a target protein, and is useful for developing and producing various target proteins for medical and industrial purposes.

### DESCRIPTION OF DRAWINGS

FIG. 1 shows an expression plasmid designed to use TRS-1, TRS-2 and EPRS as fusion proteins, in which a target protein is inserted into a multiple cloning site (MCS), and a histidine tag (6X His) for purification and a TEV protease recognition site (TEV site) for cleaving the fusion proteins and the target protein are included.

FIG. 2 is an image showing the improvement in soluble expression and the activity of GFP, caused by EPRS.

FIG. 3 is an image showing the improvement in soluble expression and the activity of interferon-beta (IFN-β), caused by EPRS.

FIG. 4 is an image showing the improvement in soluble expression and the activity of TEV protease, caused by EPRS.

FIG. 5 is an image confirming the improvement in soluble expression of a multimeric protein, caused by EPRS. In addition, an effect of increasing the expression rate of a soluble protein, caused by EPRS, was further confirmed by expressing the multimeric protein in E. coli. Heat-labile enterotoxin is a protein constituting a pentamer, and also known to be difficult to express in a soluble form in E. coli. As a result of confirming the expression of the protein fused with EPRS, it was confirmed that the EPRS-fused form showed a much higher soluble expression rate, compared to a direct form.

FIG. 6 is a schematic diagram showing a fused form of WHEP domains and ERS.

FIG. 7 is an image confirming the expression of EPRS fused with RBD and S1of COVID-19 according to one embodiment of the present invention.

FIG. 8 is an image confirming the expression of EPRS fused with RSV F-bacterioferritin according to one embodiment of the present invention.

FIG. 9 is an image confirming the expression of EPRS fused with LTB-JEVED3 according to one embodiment of the present invention.

FIG. 10 show the result of size-exclusion chromatography for TEV protease-treated EPRS-LTB.

FIG. 11 shows the result of size-exclusion chromatography for EPRS-LTB-JEVED3.

FIG. 12 shows the result of size-exclusion chromatography for TEV protease-treated EPRS-LTB-JEVED3.

### MODES OF THE INVENTION

The inventors confirmed that, among the fusion proteins including WHEP domains of hEPRS, EPRS most greatly contributes to the increase in solubility, and the present invention was completed.

The present invention provides a peptide capable of improving the solubility and folding of a target protein using a WHEP domain as a fusion partner, and an expression vector or gene construct for producing a recombinant protein.

The “vector” used herein refers to DNA that can introduce a target DNA fragment into host bacteria to proliferate in a DNA recombination experiment and is also referred to as a cloning vehicle. The vector DNA undergoes ring opening by cleavage with a restriction enzyme, and a target DNA fragment is introduced into the host bacteria by insertion and linkage to the vector DNA. The vector DNA in which the target DNA fragment is linked is replicated as the host bacteria are proliferated, and distributed to each cystic cell along with bacterial division, thereby maintaining the target DNA fragment for generations, and the vector DNA may be, for example, a plasmid or a phage chromosome.

Methods such as the selection, construction and transformation of the vector, and the expression of a recombinant protein may be easily performed by those of ordinary skill in the art to which the present invention belongs, and some modifications in conventional methods are also included in the present invention.

The “transformation” used herein refers to a genetic change in the trait of an individual or cell by DNA, which is the genetic material given from the outside.

The delivery (introduction) of the vector into a host cell may use a delivery method well known in the art. As a delivery method, for example, a microinjection method, a calcium phosphate precipitation method, an electroporation method, a liposome-mediated transfection method, or a gene bombardment method may be used, but the present invention is not limited thereto. A chemical treatment method such as PEG or a gene gun may be used.

Culture conditions in the culture of a transformant may be appropriately selected and used depending on a host cell. Conditions such as a temperature, the pH of a medium and incubation time may be appropriately adjusted to be suitable for cell growth and mass production of a protein during culture.

The host cell in the present invention may be a host cell conventionally used in the art, and preferably, E. coli.

Hereinafter, embodiments of the present invention will be described with reference to the drawings. The advantages and features of the present invention and the methods of accomplishing the same may be clearly understood with reference to the detailed description of exemplary embodiments and the accompanying drawings. However, the present invention is not limited to the exemplary embodiments disclosed below, and may be embodied in many different forms. These exemplary embodiments are merely provided to complete the disclosure of the present invention and fully convey the scope of the present invention to those of ordinary skill in the art, and the present invention should be defined by only the accompanying claims. Throughout the specification, like reference numerals denote like elements.

Unless defined otherwise, all terms (including technical and scientific terms) used herein may be used in a sense that is commonly understood by one of ordinary skill in the art to which the present invention belongs. Also, generally used predefined terms are not ideally or excessively interpreted unless explicitly defined otherwise. The terms used herein are for describing embodiments and are not intended to limit the present invention. Herein, singular forms include plural forms unless specifically stated otherwise.

### EXAMPLE 1

### Example 1-1. Construction of Protein Expression Vector

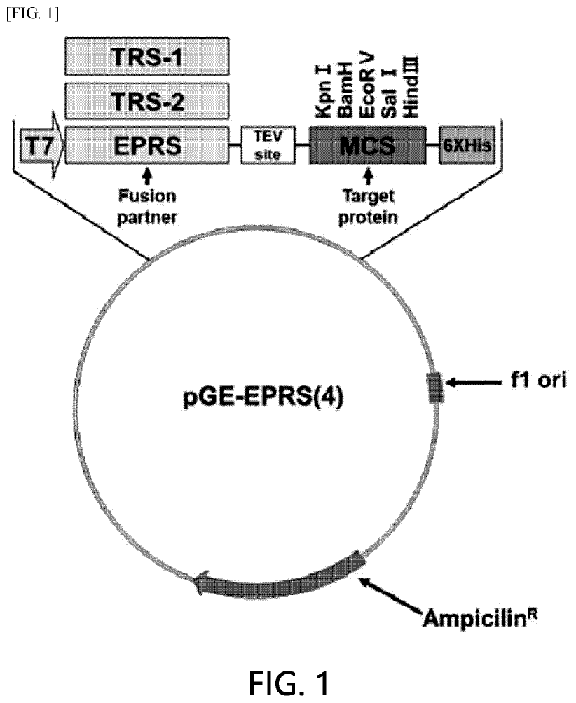

As a protein expression vector, a pGE-LysRS expression vector was used (Choi S I et al., Protein solubility and folding enhancement by interaction with RNA, PLoS ONE (2008), 3: e2677). A pGE-TRS-1, pGE-TRS-2, or pGE-EPRS expression vector was manufactured by adjusting the expression of pGE-LysRS by a T7 promoter, cleaving the LysRS gene at one of cleavage sites in Ndel and MCS (Kpn1-BamH1-EcoRV-Sal1-Hind3), and inserting TRS-1, TRS-2, or EPRS into the site. In addition, a TRS-1, TRS-2 or EPRS-fused target protein expression vector was manufactured by inserting a target protein into a C-terminus site of each expression vector (FIG. 1). As the target protein, GFP, IFN-β, TEV protease or LTB was used. The expression vector manufactured as described above is pGE-GFP, pGE-TRS-1-GFP, pGE-TRS-2-GFP, pGE-EPRS-GFP, pGE-IFN-β, pGE-EPRS-IFN-β, pGE-Tev, pGE-EPRS-Tev, pGE-LTB, or pGE-EPRS-LTB.

### Example 1-2. Protein Expression and Confirmation of Purification Pattern Through SDS-PAGE

The manufactured protein expression vector was transformed into BL21(DE3)-pLysS or SHuffle® T7 competent cells and cultured. All transformed E. coli were cultured in an LB medium containing 50 μg/mL of ampicillin, pLysS-containing E. coli was cultured in a medium supplemented with 34 μg/mL of chloramphenicol. A culture temperature is different for each protein, and culture was performed at 16 to 37° C. When the OD600 value of E. coli became 0.5 or more, 0 μM to 1 mM of IPTG was added to activate the T7 promoter, and in order to sufficiently produce a protein, after the IPTG addition, the E. coli was cultured at 16 to 37° C. for approximately 3 hours, at 25° C. for approximately 5 hours, and 16 to 24° C. for approximately 16 hours. The sufficiently cultured E. coli was centrifuged to remove a supernatant, and then stored. Afterward, 0.3 mL of PBS was added to harvested E. coli corresponding to 5 mL of the LB medium for ultrasonication, thereby obtaining a lysate. In addition, the lysate was centrifuged and divided into a soluble fraction and a pellet fraction, and each of the total lysate, the soluble fraction and the pellet fraction was analyzed by SDS-PAGE.

### Example 1-3. Protein Expression and Purification

Each of the manufactured pGE-GFP, pGE-TRS-1-GFP, pGE-TRS-2-GFP, pGE-EPRS-GFP, pGE-IFN-β, pGE-EPRS-IFN-β, pGE-Tev, and pGE-EPRS-Tev expression vectors was transformed into BL21(DE3)-pLysS competent cells and cultured. All transformed E. coli were cultured in LB containing 50 μg/mL of ampicillin and 34 μg/mL of chloramphenicol. When the OD600 value of E. coli became 0.5 or more, 1 mM IPTG was added to activate a T7 promoter, and in order to sufficiently produce a protein, after the IPTG addition, the E. coli was cultured at 30° C. for approximately 6 hours. The sufficiently cultured E. coli was centrifuged to remove a supernatant, and stored.

PBS was added to the harvested E. coli for centrifugation, thereby obtaining a lysate. In addition, the lysate was centrifuged to obtain only a soluble fraction and then purified by Ni²⁺ chromatography, and the purified protein was subjected to dialysis in PBS and concentrated using Centriprep.

Example 1-4. Measurement of GFP Activity

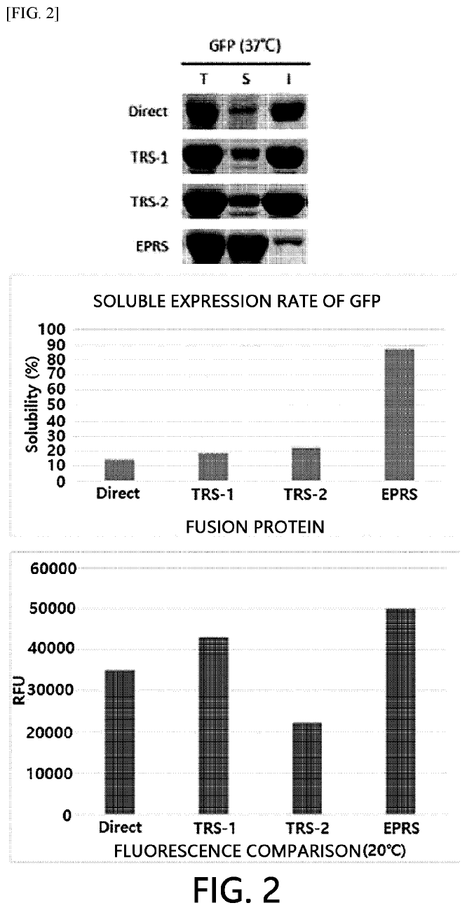

An experiment was performed to measure the activity of a GFP protein produced per unit cell. After the completion of protein expression, PBS was added to a E. coli culture from which a supernatant was removed through centrifugation, and the resulting solution was subjected to ultrasonication, thereby obtaining a lysate. Afterward, the lysate was centrifuged to obtain only a soluble fraction, and then the actual activity of GFP was measured by measuring fluorescence with a spectrophotometer (FIG. 2).

A green fluorescent protein (GFP) is known as a protein that is expressed in an insoluble form when expressed alone in E. coli. As a result of confirming the expression pattern after direct GFP expressed alone without a fusion protein and TRS-1-, TRS-2-, and EPRS-fused fusion proteins were expressed in E. coli, all of the fusion proteins showed an increase in soluble expression rate compared to the direct GFP, and it was confirmed that, particularly, the soluble expression rate of the EPRS-fused GFP was very highly increased.

As a result of measuring the activity of expressed GFP per culture dose, the TRS-2-fused GFP showed fluorescence activity similar to direct GFP, and TRS-1 and EPRS-fused GFPs showed higher fluorescent activity.

Therefore, it was confirmed that the soluble expression level of the target protein GFP is increased by TRS-1, TRS-2 and EPRS, and the produced proteins were active.

Example 1-5. Measurement of IFN-Beta Activity

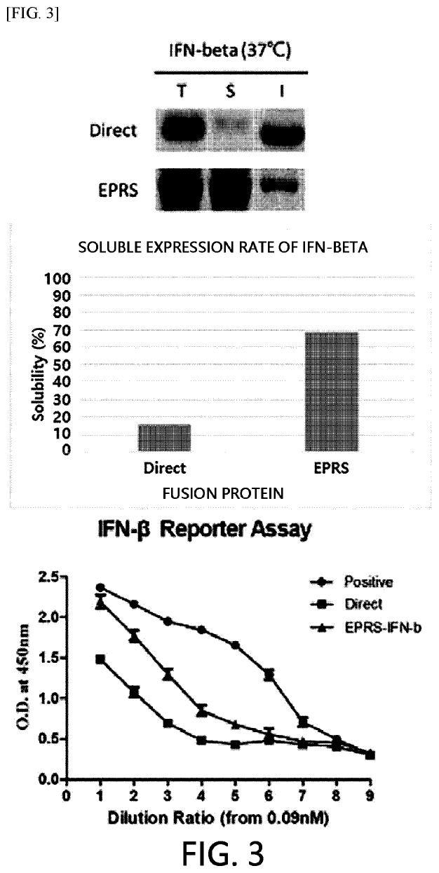

The activity of IFN-beta expressed in E. coli was measured using HEK-Blue™ IFN-α/β cells (InvivoGen). The experiment was conducted according to the seller's protocol, and it is briefly summarized as follows. 180 μL of a suspension of HEK-Blue™ IFN-α/β cells modified to observe a signaling pathway by IFN-beta and 20 μL of an IFN-beta sample were added to a 96-well plate, and incubated in an incubator (37° C., 5% CO₂) overnight. The next day, 20 μL of a supernatant in which a reaction occurred in HEK-Blue™ IFN-α/β cells and 180 μL of a QuantiBlue™ solution were mixed, and reacted at 37° C. for approximately 30 minutes to 3 hours. After the reaction was completed, a level was measured at a wavelength of 620 to 655 nm with a spectrophotometer (FIG. 3).

Interferon-beta (IFN-β) is also a protein that is expressed insoluble in E. coli, and direct IFN-β having no fusion protein shows a characteristic of a very low soluble expression rate in E. coli. When EPRS was used as a fusion protein, the fusion protein showed a high soluble expression rate.

When the activity of purified IFN-β was measured with HEK-Blue™ IFN-α/β cells (InvivoGen), the activity of commercial IFN-β expressed and purified in CHO cells was the highest, and the activity of the IFN-β fusion protein in which EPRS was fused was the next highest. Direct IFN-β showed the lowest activity.

Therefore, it was confirmed that the expression of soluble and active-form IFN-β was promoted by EPRS.

Example 1-6. Measurement of Tev Protease Activity

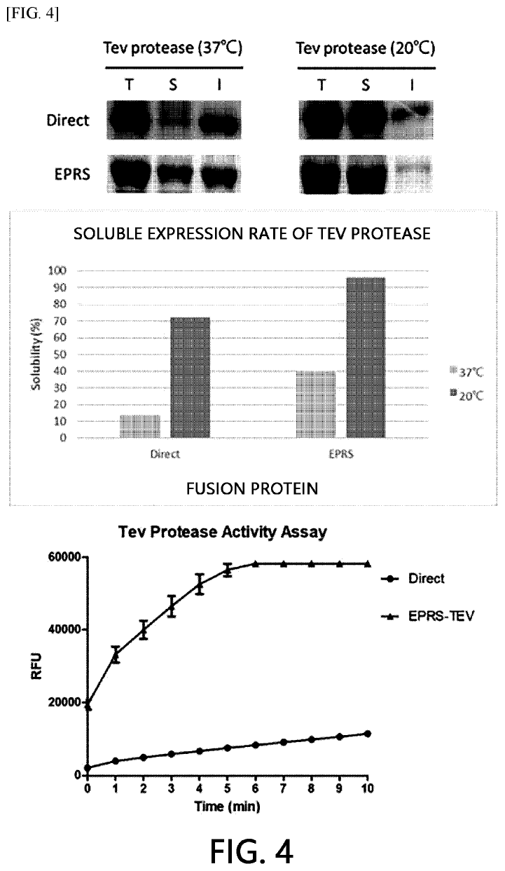

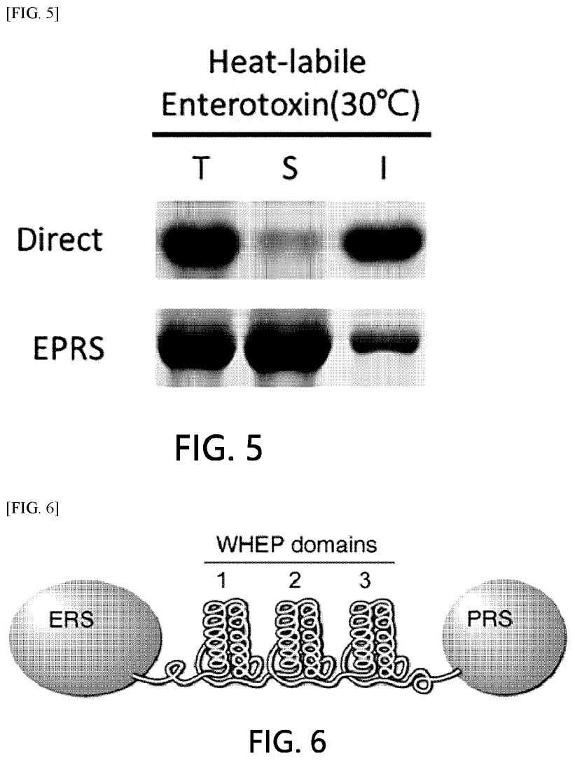

To measure the activity of TEV protease expressed in E. coli, a SensoLyte® 520 TEV Activity Assay Kit (*Fluorimetric* kit) was used. The experiment was conducted according to the seller's protocol, and simply summarized as follows. The TEV protease and 5-FAM/QXL® 520 TEV substrate for experimentation were put into a 96-well plate and fluorescence was measured every minute. Here, the measured fluorescence was Ex/Em=490/520 nm. As the substrate is cleaved by the TEV protease, fluorescence increased, and using this, the activity of the TEV protease was measured (FIG. 4).

Tobacco Etch Virus (TEV) protease is a protein with high commercial demand, and a type of protein difficult to be obtained as a recombinant protein in E. coli. When there was no fusion protein, direct TEV protease showed a low soluble expression rate in E. coli, and when EPRS was fused, an effect of increasing the soluble expression rate was shown. When an expression temperature was lowered (20° C.) in E. coli culture, both of the direct TEV protease and the EPRS-TEV protease showed an increased soluble expression rate, and particularly, the EPRS-TEV protease showed a soluble expression rate of 90% or more.

As a result of confirming the activity of purified TEV proteases with a SensoLyte® 520 TEV Activity Assay Kit, even with the same amount, the direct TEV protease showed lower activity, and the structure showing the steepest enzymatic activity was EPRS-fused EPRS-TEV.

Therefore, the soluble and active-form expression of TEV protease, caused by EPRS, was confirmed.

### EXAMPLE 2

Example 2-1. Protein Expression and Confirmation of Solubility By SDD-PAGE

Vectors manufactured using a receptor-binding domain (RBD) and relatively large spike subunit 1 (S1) of SARS-CoV-2 (COVID-19) as target proteins are pGE-EPRS-RBD and pGE-EPRS-S1.

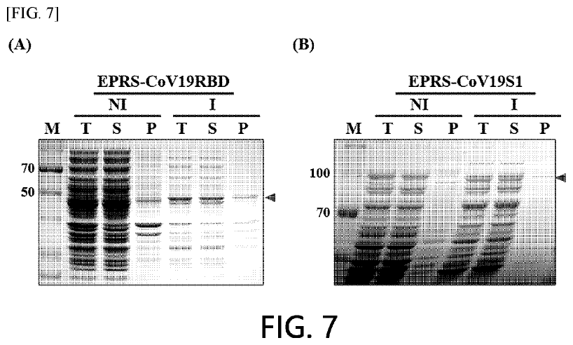

Each of the manufactured protein expression vectors was transformed into BL21 or HMS174 competent cells and cultured. All transformed E. coli were cultured in an LB medium containing 50 μg/mL of ampicillin. When the OD600 value of the E. coli became 0.5 or more, to activate a T7 promoter, 0.5 mM IPTG was added, and then incubated at 16° C. for 16 hours. The cultured E. coli was centrifuged to remove a supernatant, and stored at −80° C. Afterward, 0.3 mL of lysis buffer [50 mM Tris-Cl (pH 7.5), 300 mM NaCl, 10% glycerol, 2 mM beta-mercaptoethanol, 0.1% Tween-20] was added to the harvested E. coli corresponding to 5 mL of the LB medium and subjected to ultrasonication, thereby obtaining a lysate. In addition, the lysate was centrifuged and divided into a soluble fraction and a pellet fraction, and SDS-PAGE was used to analyze the total lysate, the soluble fraction, and the pellet fraction. The sizes of RBD and S1 were 27 kDa and 73 kDa, respectively, and the sizes of EPRS-fused RBD and S1 were 49 kDa and 95 kDa, respectively (see FIG. 7).

A vector manufactured using a respiratory syncytial virus (RSV) fusion (F) protein as a target protein and bacterioferritin as a scaffold protein for nanoparticles is pGE-EPRS-RSV F-bacterioferritin.

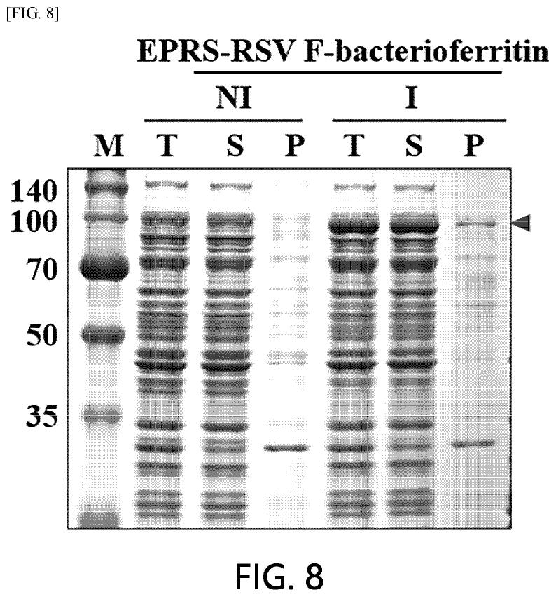

The manufactured protein expression vector was transformed into Shuffle T7 competent cells and cultured. All transformed E. coli were cultured in an LB medium containing 50 μg/mL of ampicillin. When the OD600 value of the E. coli is 0.5 or more, to activate a T7 promoter, 0.5 mM IPTG was added, and then incubated at 20° C. for approximately 6 hours. The cultured E. coli was centrifuged to remove a supernatant, and stored at −80° C. Afterward, 0.3 mL of lysis buffer [50 mM Tris-Cl (pH7.5), 200 mM NaCl, 10% glycerol, 0.1% Tween-20] was added to the harvested E. coli corresponding to 5 mL of the LB medium and subjected to ultrasonication, thereby obtaining a lysate. In addition, the lysate was centrifuged and divided into a soluble fraction and a pellet fraction, and SDS-PAGE was used to analyze the total lysate, the soluble fraction, and the pellet fraction. The size of RSV F-bacterioferritin was 70 kDa, and the size of the EPRS-fused RSV F-bacterioferritin was 92 kDa (see FIG. 8).

A vector, which is manufactured with the domain III (ED3) of an envelope protein that is present in the form of a pentamer with 5 axes in the Japanese encephalitis virus structure and is known to play an important role in immune induction as a target protein and the heat-labile toxin B subunit (LTB) as a pentameric scaffold, is pGE-EPRS-LTB-JEVED3.

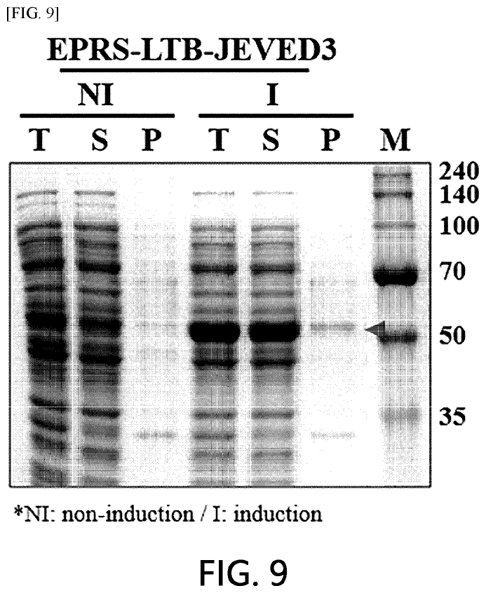

The manufactured protein expression vector was transformed into Shuffle T7 competent cells and cultured. All transformed E. coli were cultured in an LB medium containing 50 μg/mL of ampicillin. When the OD600 value of the E. coli became 0.5 or more, to activate a T7 promoter, 0.5 mM IPTG was added, and then incubated at 20° C. for approximately 6 hours. The cultured E. coli was centrifuged to remove a supernatant, and stored at −80° C. Afterward, 0.3 mL of lysis buffer [50 mM Tris-Cl(pH7.5), 300 mM NaCl, 10% glycerol] was added to the harvested E. coli corresponding to 5 mL of the LB medium and subjected to ultrasonication, thereby obtaining a lysate. In addition, the lysate was centrifuged and divided into a soluble fraction and a pellet fraction, and SDS-PAGE was used to analyze the total lysate, the soluble fraction, and the pellet fraction. The size of LTB-JEVED3 is 24 kDa, and the size of EPRS-fused LTB-JEVED3 is 46 kDa (see FIG. 9).

### Example 2-2. Protein Purification

The pGE-EPRS-LTB and pGE-EPRS-LTB-JEVED3 expression vectors transformed into Shuffle T7 competent cells were cultured in a LB medium containing 50 μg/mL of ampicillin. When the OD600 value of the E. coli became 0.5 or more, 1 mM of IPTG was added, and cultured at 20° C. for approximately 6 hours. The sufficiently cultured E. coli was centrifuged to remove a supernatant and stored at −80° C.

A buffer [50 mM Tris-Cl (pH7.5), 300 mM NaCl, 10% glycerol, 5 mM imidazole] was added to the cultured E. coli and centrifuged, thereby obtaining a lysate. In addition, the lysate was centrifuged to obtain only a soluble fraction, and then the fraction was purified by nickel chromatography. The purified protein was dialyzed in a storage buffer [50 mM Tris-Cl (pH7.5), 300 mM NaCl, 0.1 mM EDTA], and concentrated using Amicon.

### Example 2-3. Confirmation of Pentamer Through Size-Exclusion Chromatography

To verify whether the purified EPRS-LTB and EPRS-LTB-JEVED3 proteins form a pentameric structure, size-exclusion chromatography (SEC) using a Superdex 200 Increase 10/300 column (GE Healthcare) was performed, and the size of each protein was measured by calibration with a marker protein with a known size. Here, the composition of a buffer used herein is [50 mM Tris-Cl (pH7.5), 300 mM NaCl]. Both of an EPRS-fused protein and an EPRS-removed protein obtained by being treated with TEV protease to cleave a Tev cleavage site between EPRS and a target protein were verified. A fraction with a peak at the pentamer size was verified again by SDS-PAGE.

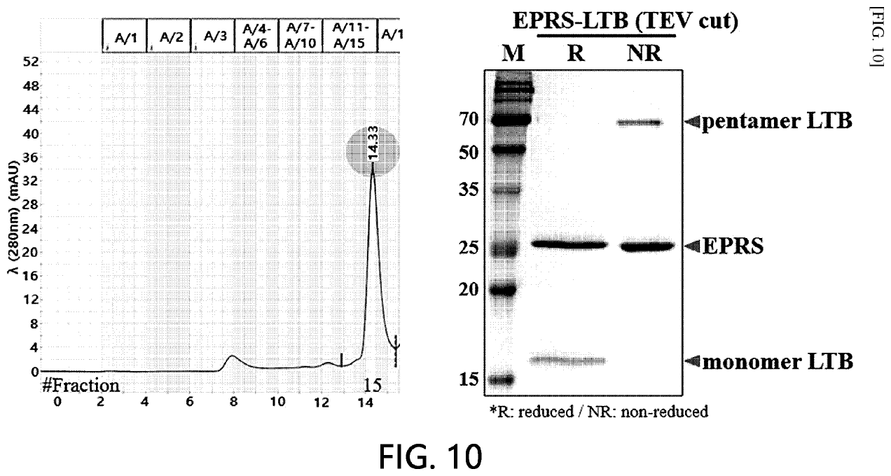

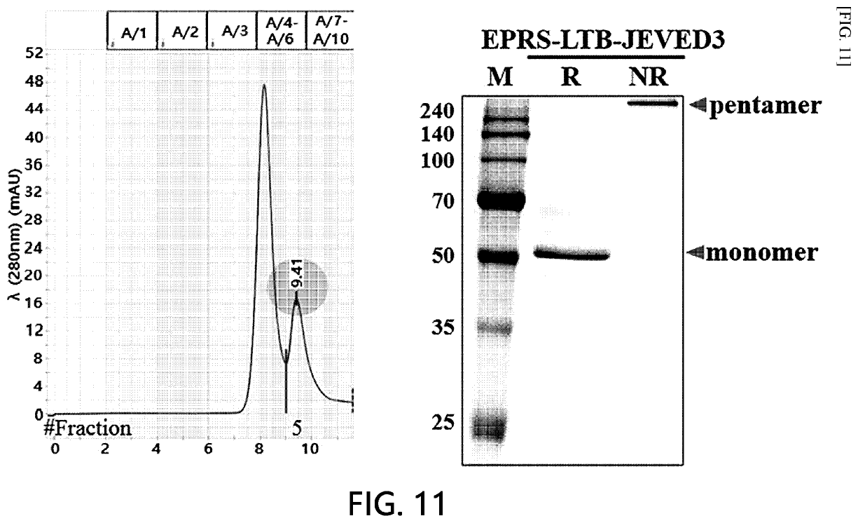

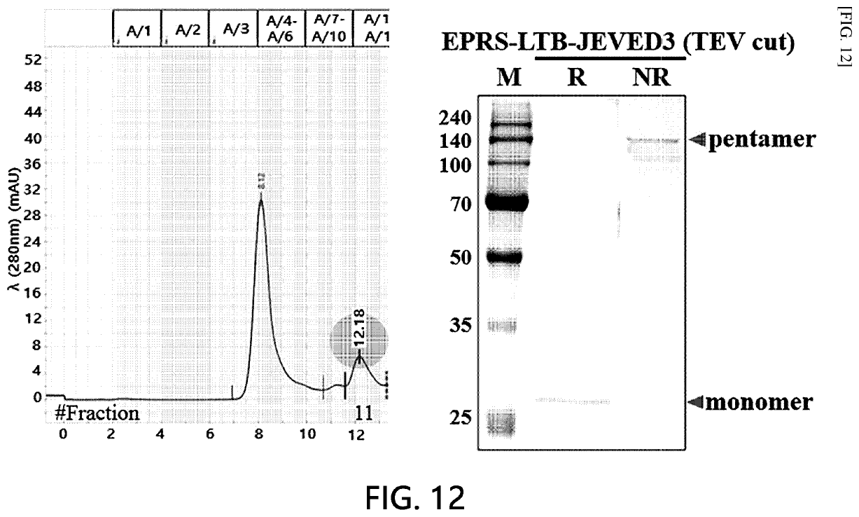

As a result, each protein showed a peak at an expected pentamer size (see FIGS. 10 to 12, left panel), and when the fraction of the peak was prepared in a reduced state with DTT and a non-reduced state without DTT and analyzed by SDS-PAGE, it was confirmed that bands of similar sizes are shown (see FIGS. 10 to 12, right panel). When the vectors were formed in a pentamer, the size of LTB is approximately 60 kDa, the size of EPRS-LTB-JEVED3 is approximately 230 kDa, and the size of LTB-JEVED3 is approximately 125 kDa. Therefore, it was confirmed that the fusion with EPRS cannot only increase the solubility of a target protein in E. coli, but also assembly into a multimer is possible by inducing suitable protein folding.

TRS-1
SEQ ID NO: 1
LYNRVAVQGDVVRELKAKKAPKEDVDAAVKQLLSLKAEYKEKTGQEYKP
GNPP
TRS-2
SEQ ID NO: 2
LYDEVAAQGEVVRKLKAEKSPKAKINEAVECLLSLKAQYKEKTGKEYIP
GQPP
TRS-3
SEQ ID NO: 3
LFDKVASQGEVVRKLKTEKAPKDQVDIAVQELLQLKAQYKSLIGVEYKP
SEQ ID NO: 4
AEIGQNISSNSSASILESKS
SEQ ID NO: 5
LSQSSDSSPTRNSEPAGLETPEAKV
EPRS sequence
SEQ ID NO: 6
LYNRVAVQGDVVRELKAKKAPKEDVDAAVKQLLSLKAEYKEKTGQEYKP
GNPPAEIGQNISSNSSASILESKSLYDEVAAQGEVVRKLKAEKSPKAKI
NEAVECLLSLKAQYKEKTGKEYIPGQPPLSQSSDSSPTRNSEPAGLETP
EAKVLFDKVASQGEVVRKLKTEKAPKDQVDIAVQELLQLKAQYKSLIGV
EYKP
COVID-19 RBD
SEQ ID NO: 7
FTVEKGIYQTSNFRVQPTESIVRFPNITNLCPFGEVFNATRFASVYAWN
RKRISNCVADYSVLYNSASFSTFKCYGVSPTKLNDLCFTNVYADSFVIR
GDEVRQIAPGQTGKIADYNYKLPDDFTGCVIAWNSNNLDSKVGGNYNYL
YRLFRKSNLKPFERDISTEIYQAGSTPCNGVEGFNCYFPLQSYGFQPTN
GVGYQPYRVVVLSFELLHAPATVCGPKKSTNLVKNKCVNFNF
COVID-19 S1
SEQ ID NO: 8
QCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLD
SKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTP
INLVRDLPQGFSALEPLVDLPIGINITRFQTLLALHRSYLTPGDSSSGW
TAGAAAYYVGYLQPRTFLLKYNENGTITDAVDCALDPLSETKCTLKSFT
VEKGIYQTSNFRVQPTESIVRFPNITNLCPFGEVFNATRFASVYAWNRK
RISNCVADYSVLYNSASFSTFKCYGVSPTKLNDLCFTNVYADSFVIRGD
EVRQIAPGQTGKIADYNYKLPDDFTGCVIAWNSNNLDSKVGGNYNYLYR
LFRKSNLKPFERDISTEIYQAGSTPCNGVEGFNCYFPLQSYGFQPTNGV
GYQPYRVVVLSFELLHAPATVCGPKKSTNLVKNKCVNFNFNGLTGTGVL
TESNKKFLPFQQFGRDIADTTDAVRDPQTLEILDITPCSFGGVSVITPG
TNTSNQVAVLYQDVNCTEVPVAIHADQLTPTWRVYSTGSNVFQTRAGCL
IGAEHVNNSYECDIPIG
Heat-labile toxin B subunit(LTB)
SEQ ID NO: 9
APQTITELCSEYRNTQIYTINDKILSYTESMAGKREMVIITFKSGETFQ
VEVPGSQHIDSQKKAIERMKDTLRITYLTETKIDKLCVWNNKTPNSIAA
ISMKN
Bacterioferritin
SEQ ID NO: 10
MKGDTKVINYLNKLLGNELVAINQYFLHARMFKNWGLKRLNDVEYHESI
DEMKHADRYIERILFLEGLPNLQDLGKLNIGEDVEEMLRSDLALELDGA
KNLREAIGYADSVHDYVSRDMMIEILRDEEGHIDWLETELDLIQKMGLQ
NYLQAQIREEG
RSV F
SEQ ID NO: 11
QNITEEFYQSTCSAVSKGYLSALRTGWYTSVITIELSNIKENKCNGTDA
KVKLIKQELDKYKNAVTELQLLMQSTPPTNNRARRGGSGGSGGSGFLGF
LLGVGSAIASGVAVCKVLHLEGEVNKIKSALLSTNKAVVSLSNGVSVLT
FKVLDLKNYIDKQLLPILNKQSCSISNIETVIEFQQKNNRLLEITREFS
VNAGVTTPVSTYMLTNSELLSLINDMPITNDQKKLMSNNVQIVRQQSYS
IMCIIKEEVLAYVVQLPLYGVIDTPCWKLHTSPLCTTNTKEGSNICLTR
TDRGWYCDNAGSVSFFPQAETCKVQSNRVFCDTMNSLTLPSEINLCNVD
IFNPKYDCKIMTSKTDVSSSVITSLGAIVSCYGKTKCTASNKNRGIIKT
FSNGCDYVSNKGMDTVSVGNTLYYVNKQEGKSLYVKGEPIINFYDPLVF
PSDEFDASISQVNEKINQSLAFIRKSDELL
JEV Envelope domain III(ED3)
SEQ ID NO: 12
KGTTYGMCTEKFSFAKNPADTGHGTVVIELSYSGSDGPCKIPIVSVASL
NDMTPVGRLVTVNPFVATSSANSKVLVEMEPPFGDSYIVVGRGDKQINH
HWHKAG

1. A peptide for enhancing the expression efficiency of a target protein, comprising the sequence of a domain isolated from human glutamyl-prolyl tRNA synthetase (hEPRS).

2. The peptide of claim 1, wherein the domain includes TRS-1 (SEQ ID NO: 1) and TRS-2 (SEQ ID NO: 2).

3. The peptide of claim 2, wherein the domain further includes TRS-3 (SEQ ID NO: 3).

4. The peptide of claim 1, further comprising linkers for connecting the sequences of the domains.

5. The peptide of claim 4, wherein the linker sequence is represented by SEQ ID NO: 4 or 5.

6. The peptide of claim 1, further comprising a tag sequence for improving the expression efficiency of a target protein.

7. The peptide of claim 1, wherein the target protein includes one or more selected from the group consisting of an antigen, an antibody, a cell receptor, an enzyme, a structural protein, serum, and a cell protein.

8. The peptide of claim 7, wherein the antigen includes one or more selected from the group consisting of novel coronavirus (SARS-CoV-2), the hemagglutinin (HA) of influenza virus, respiratory syncytial virus (RSV), and Nipah virus.

9. A polynucleotide encoding the peptide according to claim 1.

10. An expression vector, comprising:
a polynucleotide encoding a target protein; and
a polynucleotide encoding a peptide according to claim 1 binding to the 5′-end of the polynucleotide encoding the target protein.

11. A host cell transformed with the expression vector of claim 10.

12. The host cell of claim 11, which is E. coli.

13. A method of producing a soluble target protein, comprising:
(A) preparing an expression vector including a polynucleotide encoding a target protein and a nucleotide encoding the peptide of claim 1 binding to the 5′ end of the polynucleotide;
(B) preparing a transformant by introducing the expression vector into host cells; and
(C) culturing the transformant to induce the expression of a recombinant target protein and obtaining the target protein.
